(19) United States
(12) Patent Application Publication
(10) Pub. No.: US 2018/0271836 A1
(43) Pub. Date: Sep. 27, 2018

(54) TOPICAL NON-AQUEOUS PHARMACEUTICAL FORMULATIONS CONTAINING AN ALKYL SULFOXIDE FOR THE TREATMENT OF TOPICAL REFERENCE INFECTIONS

(71) Applicant: Samy Saad, Mississauga (CA)

(72) Inventor: Samy Saad, Mississauga (CA)

(21) Appl. No.: 15/990,727

(22) Filed: May 28, 2018

Related U.S. Application Data
(63) Continuation of application No. 13/798,366, filed on Mar. 13, 2013.

(30) Foreign Application Priority Data
May 2, 2012 (CA) 2775393

(51) Int. Cl. A61K 31/4196; A61K 47/10; A61K 47/20; A61K 9/00; A61K 47/38

(52) U.S. Cl. CPC A61K 31/4196; A61K 47/10; A61K 47/20; A61K 47/38; A61K 9/0014

(57) ABSTRACT

The present application relates to non-aqueous pharmaceutical formulations containing an anti-microbial agent.

### CROSS TO RELATED APPLICATION

This application is a U.S. Continuation which claims priority to U.S. patent application Ser. No. 13/798,366, filed on Mar. 13, 2013 and claims priority from Canadian Patent no. 2,775,393, issued Apr. 29, 2014, the disclosures of which are incorporated by reference herein.

### FIELD

The present application relates to preservative-free non-aqueous pharmaceutical formulations containing an anti-microbial agent.

### INTRODUCTION

Topical infections of the body, such as bacterial and/or fungal infections, can be difficult to treat as the result of systemic medications failing to reach the site of infection, or failing to reach a minimum inhibitory concentration at the site of infection, resulting in failure to treat the infection.

Topical pharmaceutical formulations are well-known, but suffer from many drawbacks. For example, fungal infections of the unguis (nail bed) are difficult to treat topically, as the anti-fungal agent cannot easily penetrate the nail cornified structure in order to reach the underlying infection.

### SUMMARY

The present disclosure relates to pharmaceutical formulations containing an anti-microbial agent which effectively treats topical infections, such as bacterial, viral or fungal infections.

In one embodiment of the disclosure, the formulation comprises a non-aqueous topical pharmaceutical formulation comprising:
(a) an anti-microbial pharmaceutical agent present in an amount between 0.01% and 80% by weight of the total formulation;
(b) an organic solvent with tissue-permeation ability present in an amount between 1% and 20% by weight of the total formulation;
(c) an organic co-solvent present in an amount between 3% and 30% by weight of the total formulation; and
(d) a film forming agent present in an amount between 10% and 85% by weight of the total formulation.

In one embodiment of the disclosure, the formulation comprises a non-aqueous topical pharmaceutical formulation consisting of:
(a) an anti-microbial pharmaceutical agent present in an amount between 0.01% and 80% by weight of the total formulation;
(b) an organic solvent with tissue-permeation ability present in an amount between 1% and 20% by weight of the total formulation;
(c) an organic co-solvent present in an amount between 3% and 30% by weight of the total formulation; and
(d) a film-forming agent present in an amount between 10% and 85% by weight of the total formulation.

In one embodiment, the non-aqueous topical pharmaceutical formulation is used for the treatment of a topical tissue infection without desquamation of the tissue.

In another embodiment, the anti-microbial agent is an anti-fungal agent, an antibiotic or an antiseptic. In another embodiment, the anti-fungal agent is an allylamine, an imidazole or a triazole, or an antibiotic. In a further embodiment, the anti-fungal agent is Fluconazole, Tolnaftate, Miconazole, Clotrimazole, Tioconazole, Nystatin, Terconazole, Butoconazole nitrate, Undecylenic acid, Clioquinol, Ciclopirox, Olamine, Econazole nitrate, Triacetin, Flucytosine, Terbinafine or Ketoconazole. In one embodiment, the anti-fungal agent is Fluconazole.

In another embodiment of the disclosure, the skin permeation agent is an aprotic solvent, such as a C₁-C₁₀-alkyl sulfoxide, for example, dimethyl sulfoxide.

In one embodiment, the organic solvent is a glycol or a polyglycol. In another embodiment, the organic solvent is ethoxydiglycol, butylene glycol, hexylene glycol or dipropylene glycol. In a further embodiment, the organic solvent is ethoxydiglycol.

In another embodiment of the disclosure, the film forming agent is a collodion. In a further embodiment, the film forming agent is flexible collodion.

In another embodiment of the disclosure, the pharmaceutical formulation consists of:
(a) an anti-microbial pharmaceutical agent present at an amount between 1% and 20% by weight of the total formulation;
(b) an organic solvent with tissue-permeation ability present at an amount between 10% and 20% by weight of the total formulation;
(c) an organic co-solvent present at an amount between 3% and 15% by weight of the total formulation; and
(d) a film forming agent present at an amount between 40% and 80% by weight of the total formulation.

In another embodiment, the formulation is applied once, or optionally twice, to the infection site in a 24-hour period, thereby delivering the active antimicrobial over a sustained period of time, favoring patient adherence to treatment, preventing relapse and facilitating recovery from the treated infection.

In another embodiment of the disclosure, there is also included a pharmaceutical formulation consisting of:
(a) an anti-fungal agent;
(b) dimethylsulfoxide;
(c) ethoxydiglycol; and
(d) flexible collodion
wherein the non-aqueous topical pharmaceutical formulation is used for the treatment of a topical tissue infection without desquamation of the tissue.

In a further embodiment, the pharmaceutical formulation consists of:
(a) an anti-fungal agent, such as a triazole, for example, fluconazole, present at an amount of 10% by weight of the total formulation;
(b) dimethylsulfoxide present at an amount of 15% by weight of the total formulation;
(c) ethyldiglycol present at an amount of 10% by weight of the total formulation; and
(d) flexible collodion present at an amount of 65% by weight of the total formulation.

In another embodiment, the infection is a fungal infection, a viral infection or a bacterial infection. In another embodiment, the fungal infection is an ungual infection, such as an Onchomycosis infection.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description

### BRIEF DESCRIPTION OF THE DRAWINGS

The disclosure will now be described in greater detail with reference to the following drawings in which:

FIG. 1 shows photographs demonstrating (a) an infected unguis of the fingers; (b) unguis subsequent to treatment with a formulation of the present disclosure;

FIG. 2 shows photographs demonstrating (a) an infected unguis of the toes; (b) unguis subsequent to treatment with a formulation of the present disclosure

### DESCRIPTION OF VARIOUS EMBODIMENTS

### (I) Definitions

The term “non-aqueous” as used herein refers to a pharmaceutical formulation that is substantially free, or totally free of water. Accordingly, water does not form a component of the formulations of the disclosure, though there may be residual water present in any of the other components.

The term “anti-microbial pharmaceutical agent” as used herein refers to any pharmacologically active agent which is used to topically treat infections, such as bacterial infections, fungal infections, protozoan infections and/or viral infections. Accordingly, the term includes any substance which kills or inhibits the growth of microorganisms such as bacteria, fungi, protozoa or viruses on the skin or exterior of a human or animal.

The term “organic solvent with tissue permeation ability” as used herein refers to any organic solvent which is able to dissolve the pharmaceutical agent and act as a carrier to deliver the pharmaceutical agent through the skin or unguis of a human or animal to the topical site of the infection. For example, the skin permeation agent is a charged compound or an aprotic solvent, which possesses the ability to dissolve the pharmaceutical agent and also to penetrate the skin or unguis at the site of infection to deliver the agent to the infection.

The term “organic co-solvent” as used herein refers to any solvent which is able to help to solubilize the active pharmaceutical agent. Examples of organic co-solvents include polyglycols, alcohols or mixtures thereof. In one embodiment, the organic co-solvent also possesses inherent broad-spectrum antiseptic or anti-microbial properties, and therefore, preservatives are not required for the pharmaceutical formulations of the disclosure making them self-preserved.

The term “film forming agent” as used herein refers to any compound which has the ability to form a moisture-resistant film or barrier after application of the pharmaceutical formulation to the site of infection. For example, flexible collodion which is one example of a film forming agent, dries after application to form a transparent film over the site of application.

The term “desquamation” as used herein refers to the shedding, burning, descaling, peeling, etc. of the outermost layer of skin and/or unguis of a human or mammal. In one embodiment, the components of the formulations of the present disclosure do not cause a desquamation of the skin and/or unguis at the site of topical tissue infection.

The phrase “therapeutically effective amount” when used herein in connection with the formulations containing active agents, means that amount of pharmaceutical agent, which provides a therapeutic benefit in the prevention, treatment, or management, of a topical infection, or one or more symptoms thereof. Different therapeutically effective amounts may be applicable for each infection, as will be readily known or determined by those of ordinary skill in the art.

### (II) Formulations

The present disclosure relates to non-aqueous topical pharmaceutical formulations. In one embodiment, the pharmaceutical formulations are for the treatment of topical infections, such as bacterial infections or fungal infections, for example ungual fungal infections, in which the formulations are able to effectively treat the infections without desquamation of the skin and/or unguis. Many pharmaceutical formulations for the treatment of topical skin and/or nail infections, include a desquamating agent which weakens or destroys the natural skin/nail barrier so that the pharmaceutical agent is effectively delivered to the site of the infection. For example, an Onchomycosis infection is a fungal infection of the nail bed, nail plate or both, which has been treated with topical anti-fungal formulations, which contain an anti-fungal agent, and in addition to other components, a desquamating agent, which corrode the unguis so that the anti-fungal agent can be delivered to the site of infection underneath the nail. Without the presence of a desquamating agent, the nail prevents the anti-fungal agent from being delivered to the site of infection, and accordingly, the Onchomycosis is not effectively treated. Accordingly, the non-aqueous topical pharmaceutical formulations of the present disclosure are able to effectively treat a topical infection without desquamating the tissue at the site of the infection.

In one embodiment, the non-aqueous topical pharmaceutical formulations of the present disclosure comprise one or more anti-microbial pharmaceutical agents, an organic solvent with tissue-permeating ability, an organic co-solvent, and a film forming agent, wherein the non-aqueous topical pharmaceutical formulation is used for the treatment of a topical tissue infection without desquamation of the tissue.

In one embodiment, all of the components of the non-aqueous pharmaceutical formulations of the disclosure have anti-microbial or antiseptic properties, resulting in a formulation that is self-preserved and provides a synergistic combination to minimize resistance of the infection to topical treatment, to rapidly control the infection and shorten the time to healing. Moreover, as the formulations are non-aqueous and the components chosen have inherent antiseptic or anti-microbial properties, the formulations do not require a preservative to inhibit the growth of certain pathogenic microorganisms. On the contrary, aqueous based formulations require a preservative to inhibit pathogenic microorganisms which can flourish in aqueous-based solutions, reducing the shelf-life of the final formulation. In addition, as the pharmaceutical formulations of the present disclosure are highly effective topical formulations for the treatment of infections, the active agents exhibit minimal systemic absorption, thereby minimizing systemic toxicity and minimizing potential interactions with other systemic medications. Finally, the topical pharmaceutical formulation includes a film forming agent, which forms a non-occlusive membrane over the infected area and therefore, the formulation need only be applied once a day (though it can be applied several times daily if necessary), which helps with patient adherence to the treatment regimen, shortening time to healing and preventing relapse of treatment.

In one embodiment, the anti-microbial agent is any substance possessing anti-bacterial, anti-fungal, anti-protozoan or anti-viral properties. In one embodiment, antibacterial agents include Acrosoxacin, Amifloxacin, Amoxycillin, Ampicillin, Aspoxicillin, Azidocillin, Azithromycin, Aztreonam, Balofloxacin, Benzylpenicillin, Biapenem, Brodimoprim, Cefaclor, Cefadroxil, Cefatrizine, Cefcapene, Cefdinir, Cefetamet, Cefmetazole, Cefprozil, Cefroxadine, Ceftibuten, Cefuroxime, Cephalexin, Cephalonium, Cephaloridine, Cephamandole, Cephazolin, Cephradine, Chlorquinaldol, Chlortetracycline, Ciclacillin, Cinoxacin, Ciprofloxacin, Clarithromycin, Clavulanic Acid, Clindamycin, Clofazimine, Cloxacillin, Danofloxacin, Dapsone, Demeclocycline, Dicloxacillin, Difloxacin, Doxycycline, Enoxacin, Enrofloxacin, Erythromycin, Fleroxacin, Flomoxef, Flucloxacillin, Flumequine, Fosfomycin, Isoniazid, Levofloxacin, Mandelic Acid, Mecillinam, Metronidazole, Minocycline, Mupirocin, Nadifloxacin, Nalidixic Acid, Nifuirtoinol, Nitrofurantoin, Nitroxoline, Norfloxacin, Ofloxacin, Oxytetracycline, Panipenem, Pefloxacin, Phenoxymethylpenicillin, Pipemidic Acid, Piromidic Acid, Pivampicillin, Pivmecillinam, Prulifloxacin, Rufloxacin, Sparfloxacin, Sulbactam, Sulfabenzamide, Sulfacytine, Sulfametopyrazine, Sulphacetamide, Sulphadiazine, Sulphadimidine, Sulphamethizole, Sulphamethoxazole, Sulphanilamide, Sulphasomidine, Sulphathiazole, Temafloxacin, Tetracycline, Tetroxoprim, Tinidazole, Tosufloxacin, Trimethoprim and salts or esters thereof. In another embodiment, the antifungal agents include Bifonazole, Butoconazole, Chlordantoin, Chlorphenesin, Ciclopirox Olamine, Clioquinol, Clotrimazole, Eberconazole, Econazole, Fluconazole, Flucyosine, Flutrimazole, Isoconazole, Itraconazole, Ketoconazole, Miconazole, Nifuroxime, Nystatin, Olamine, Tioconazole, Tolnaftate, Terconazole, Triacetin, Undecenoic Acid, Unecylenic acid and salts or esters thereof. In another embodiment, the antiprotozoal agents include Acetarsol, Azanidazole, Chloroquine, Metronidazole, Nifuratel, Nimorazole, Omidazole, Propenidazole, Secnidazole, Sineflngin, Tenonitrozole, Temidazole, Tinidazole and salts or esters thereof. In a further embodiment, antiviral agents include Acyclovir, Brivudine, Cidofovir, Curcumin, Desciclovir, 1-Docosanol, Edoxudine, Fameyclovir, Fiacitabine, Ibacitabine, Imiquimod, Lamivudine, Penciclovir, Valacyclovir, Valganciclovir and salts or esters thereof.

In another embodiment, the anti-microbial agent is a silver-containing compound, and includes any compound which delivers silver ions to act as the anti-microbial agent. In one embodiment, the silver-containing compound is silver sulfadiazine, a silver salt such as silver nitrate, silver zeolite or silver nanoparticles. In another embodiment, the silver-containing antimicrobial agent is silver sulfadiazine.

In another embodiment, the anti-microbial agent is a boron-containing compound. In one embodiment, the boron-containing compound is a diazaborine, an N-sulfonyl diazaborine, a peptide boronic acid derivative, and/or boronic acid derivatives. Other boron-containing compounds include those described in Baker, S J., et al., Journal of Medicinal Chemistry, Volume 49, Number 15, pp. 4447-4450.

In another embodiment, the anti-microbial agent is a mercury-containing compound, such as Merbromine and/or its derivatives.

In one embodiment, the anti-microbial agent is present in an amount between 0.01% and 80% by weight of the total formulation, optionally between 0.1% and 50%, or 0.1% and 20%, optionally 1% and 20%. It will be understood that a person skilled in the art will be able to determine the appropriate amount of anti-microbial agent for a formulation depending on the type of infection, the anti-microbial agent used, the severity of the infection and the age of the patient being treated, etc.

In one embodiment, the pharmaceutical formulations of the disclosure comprise an organic solvent with tissue-permeation ability, which are able to dissolve the one or more anti-microbial pharmaceutical agents. Accordingly, as the anti-microbial pharmaceutical agents are of a wide chemical structural variety, the skin permeation agent is able to dissolve both lipophilic and/or hydrophilic pharmaceutical agents. In addition, the skin permeation agent is able to penetrate the skin or unguis (for example, a nail) of a human or animal, while simultaneously delivering the pharmaceutical agent through the skin or unguis to the site of infection under the skin or unguis.

In one embodiment, the organic solvent with tissue permeation ability, or skin permeation agent, is an aprotic solvent, such as dimethylformamide, acetone, or a C₁-C₁₀-alkyl sulfoxide. In one embodiment, the C₁-C₁₀-alkyl sulfoxide is dimethyl sulfoxide. In addition, in one embodiment, the skin permeation agent also possesses anti-inflammatory, anti-pruritic and anti-infective properties, which aid in the healing of the infection. In another embodiment, the skin permeation agent is present in the formulations in an amount between about 1% and 20% by weight of the total formulation, optionally 3% to 20%, optionally 10% and 20%, optionally about 15%. The amount of skin permeation agent needed in each formulation will be dependent upon the desired viscosity of the formulation, as well as the desired rate of evaporation and the rate of penetration into the anatomical structure (e.g. skin or nail). In one embodiment, using a higher amount of the skin permeation agent in the final formulation will result in a formulation having a lower viscosity, a higher rate of evaporation and a higher rate of penetration across the anatomical structure. In another embodiment, the skin permeation agent obviates the need for a corrosive (e.g. a metal hydroxide) or keratolytic agent (e.g. urea, benzoylperoxide, salicylic acid, resorcinol, tretinoin) which acts by the desquamation or removal of the upper layers of the diseased infection site (e.g. nail). In one embodiment, the pharmaceutical formulations of the present disclosure do not contain corrosive or keratolytic agents, thereby preserving the integrity of the anatomical structure being treated and not causing disfigurement or chemical trauma to that structure.

In another embodiment, the organic solvent with tissue permeation abilities (or skin permeation agent) is present at a concentration which does not result in systemic toxicity to the patient, while still effectively treating the infection. For example, in one embodiment, when the skin permeation agent is dimethyl sulfoxide (DMSO), higher concentrations may result in undesirable side-effects such as garlic-like odor. Accordingly, the skin permeation agents of the present disclosure are present in an amount between about 1% and 20% by weight of the total formulation, or 3% and 20%, optionally 10% to 20%, optionally about 15%.

In another embodiment of the disclosure, the organic co-solvent helps to solubilize the pharmaceutical agent, and also acts as a carrier and a stabilizer of the agent. In one embodiment, the organic solvent comprises a polyglycol or an alcohol. In another embodiment, the organic solvent comprise a compound having at least one free hydroxy group, for example one hydroxy group or two hydroxy groups, such as ethoxydiglycol, butylene glycol, hexylene glycol and dipropylene glycol. The average molecular weight of the glycol solvents is between about 100 to about 500, optionally about 100 to about 250 g/mol. As described above, the organic co-solvent also possesses antiseptic and/or anti-microbial properties, which enhances the anti-microbial activity of the anti-microbial pharmaceutical agent. In one embodiment, the organic co-solvent is ethoxydiglycol, which is believed to cause genetic mutations in fungi, therefore leading to enhanced efficacy, reduction of resistance to treatment and faster healing times of a topical fungal infection. In one embodiment, the alcoholic solvent is present in the formulations in an amount between about 3% and 30%, optionally 3% to 15%, by weight of the total formulation. In one embodiment, the amount of organic solvent present in the formulation will depend on the desired final viscosity of formulation and the desired rate of evaporation.

In one embodiment of the disclosure, the film forming agent is included in the formulations to form a flexible film over the site of infection after the topical application of the formulation. In one embodiment, the film forming agent is flexible collodion. In another embodiment, the film forming agent is miscible with the skin permeation agent and the organic solvent. The film forming agent forms a moisture-resistant film that adheres to the infected site after topical application, forming a non-occlusive membrane which allows the formulation to continue to deliver the anti-microbial pharmaceutical agent over a sustained time, eliminating the need for multiple applications, thereby enhancing adherence to treatment which leads to faster recovery. In addition, the film forming agent in the non-aqueous pharmaceutical formulation protects the infected site from moisture and humidity, as fungi in the case of fungal infections, are more difficult to treat in humid conditions. In one embodiment, the film forming agent is strong enough to protect against moisture and humidity incursion, yet flexible enough that the dried film is able to be peeled away by the patient before the next application of the pharmaceutical formulation, without any need to use a removing agent such as nail-polish remover, thereby enhancing adherence and speeding-up recovery. In one embodiment, the film forming agent is dissolved in an organic solvent, such as diethyl ether, which evaporates upon application resulting the formation of the film. In one embodiment, the organic solvent which dissolves the film forming agent also possesses selective inherent mutagenic properties against fungi, which therefore enhances the activity of the formulation and also helps to preserve the formulation, rendering it self-preserved. In one embodiment, the film forming agent is present in an amount between about 10% and 85% by weight of the total formulation. In one embodiment, the amount of film forming agent in the formulations will be dependent upon the desired viscosity of the final formulation and the desired thickness of the film after application of the formulation. In one embodiment, the higher the concentration of the film forming agent, the higher the viscosity and the thickness of the film. In another embodiment, at a concentration of about 40% to about 80%, optionally 60% to about 70% of the film forming agent, the formulation after application results in a film upon drying that adheres to the site of infection, for example a nail, and acts as a non-occlusive membrane that allows the anti-microbial agent to be continuously delivered across the site of infection for a sustained-release effect, while protecting the infection from moisture and humidity.

In one embodiment of the disclosure, the formulation comprises a non-aqueous topical pharmaceutical formulation consisting of:
(a) an anti-microbial pharmaceutical agent present in an amount between 0.01% and 80% by weight of the total formulation;
(b) an organic solvent with tissue-permeation ability present in an amount between 1% and 20% by weight of the total formulation;
(c) an organic co-solvent present in an amount between 3% and 30% by weight of the total formulation; and
(d) a film forming agent present in an amount between 10% and 85% by weight of the total formulation.

In another embodiment, the anti-microbial agent is an anti-fungal agent, such as an allyl amine, an imidazole or a triazole, or an antibiotic. In a further embodiment, the anti-fungal agent is Fluconazole, Tolnaftate, Miconazole, Clotrimazole, Tioconazole, Nystatin, Terconazole, Butoconazole nitrate, Undecylenic acid, Clioquinol, Ciclopirox, Olamine, Econazole nitrate, Triacetin, Flucytosine, Terbinafine or Ketoconazole. In one embodiment, the anti-fungal agent is Fluconazole.

In another embodiment of the disclosure, the organic solvent with tissue permeation ability, or skin permeation agent, is a C₁-C₁₀-alkyl sulfoxide, such as dimethyl sulfoxide.

In one embodiment, the organic co-solvent is ethoxydiglycol, butylene glycol, hexylene glycol or dipropylene glycol. In a further embodiment, the organic solvent is ethoxydiglycol.

In another embodiment of the disclosure, the film forming agent is a collodion. In a further embodiment, the film forming agent is flexible collodion.

In another embodiment of the disclosure, the pharmaceutical formulation consists essentially of:
(a) an anti-microbial active pharmaceutical agent;
(b) an organic solvent with tissue-permeation ability;
(c) an organic co-solvent such as a glycolic solvent; and
(d) a film forming agent.

In another embodiment of the disclosure, the pharmaceutical formulation consists essentially of:
(a) an anti-microbial active pharmaceutical agent present at an amount of between about 1% and 20% by weight of the total formulation;
(b) an organic solvent with tissue-permeation ability present at an amount of between about 10% and 20% by weight of the total formulation;
(c) an organic co-solvent such as alcoholic solvent present at an amount of between about 3% and 15% by weight of the total formulation; and
(d) a film forming agent present at an amount of between about 40% and 80% by weight of the total formulation

In another embodiment of the disclosure, the pharmaceutical formulation consists of:
(a) an anti-microbial active pharmaceutical agent present at an amount of between about 1% and 20% by weight of the total formulation;
(b) an organic solvent with tissue-permeation ability present at an amount of between about 10% and 20% by weight of the total formulation;
(c) an organic co-solvent such as alcoholic solvent present at an amount of between about 3% and 15% by weight of the total formulation; and
(d) a film forming agent present at an amount of between about 40% and 80% by weight of the total formulation.

In another embodiment of the disclosure, there is also included a pharmaceutical formulation consisting of:
(a) an anti-fungal agent;
(b) dimethylsulfoxide;
(c) ethyldiglycol; and
(d) flexible collodion.

In a further embodiment, the pharmaceutical formulation consists of:
(a) an anti-fungal agent, such as a triazole, for example, fluconazole, present at an amount of 10% by weight of the total formulation;
(b) dimethylsulfoxide present at an amount of 15% by weight of the total formulation;
(c) ethyldiglycol present at an amount of 10% by weight of the total formulation; and
(d) flexible collodion present at an amount of 65% by weight of the total formulation.

In another embodiment of the disclosure, there is also included a non-aqueous formulation, comprising:

an organic solvent with tissue permeation ability;

an alcoholic solvent; and

a film forming agent.

In one embodiment, the formulation containing an organic solvent with tissue permeation function or ability, an organic co-solvent and a film forming agent form a liquid base that allows a person skilled in the art to include in the base most active pharmaceutical agents which are suitable for topical administration. For example, steroids for the formulation of medicaments for the treatment of eczema are formulated using this formulation. In one embodiment, the non-aqueous formulation, consists of a organic solvent with tissue permeation ability (skin permeation agent), an organic co-solvent and a film forming agent. In another embodiment, the non-aqueous formulation consists of dimethylsulfoxide, ethoxydiglycol and flexible collodion.

In an another embodiment of the disclosure, there is also included a method of treating a topical infection, comprising:
(a) applying a pharmaceutical formulation of the present disclosure; and
(b) allowing the pharmaceutical formulation to dry;
   wherein the pharmaceutical formulation needs only to be applied once a day, optionally twice a day, and repeating steps (a) and (b) until the infection has been successfully treated.

In one embodiment, the method of treatment may be used alone or in conjunction with other anti-microbial treatments for different indications. For example, at the same time a patient is being treated using the topical composition of this invention, the patient may also be taking oral anti-infectives to treat other conditions systemically. An advantage of treating the patient's fungal infection topically using this invention that it allows other systemic anti-infectives to be administered systemically with no contraindication as a result of drug-drug interaction between the systemic anti-infective and the systemic antifungal (such as fluconazole and terbinafine) if the patient were to be treated systemically for the fungal infection.

In one embodiment, in applying other formulations to the site of infection, the entire surface of the infection, for example the nail, is covered. Optionally, the formulation, once applied to the site of infection, is covered by a covering material that will aid in keeping the formulation in place for the period of time desired, though this is not required. The covering may be occlusive or semi-occlusive, but will be of nature that will retain the formulation. Thus, a simple bandage which has adhesive arms that will stick to the skin or nail and has a covering area that will cover the entire site is useful. An advantage of the current disclosure over prior art is that applying an occlusive dressing to the site of infection is not required for proper treatment.

In one embodiment, the formulation may be stored in a bottle or tube and applied by squeezing the composition onto the site of infection or it may be brushed on to the site using a brush and a suitable container, or with a self-dropper. Alternatively, a prepackaged single application dose may also be used where the amount for a single application is retained in a device.

In another embodiment, once the formulation is on the site of infection it is retained there for an appropriate length of time that will depend on the concentration of the active ingredient in the formulation and the individual patients' requirements. The formulation may be kept on for a shorter period of time if a higher concentration of the active ingredient is employed and is kept on for a longer period of time if a lower concentration is used. Generally, the formulation will be kept on for about 24 hours, at which point the formulation is easily removed by simple peeling or washing without any need to apply any other chemical such as nail varnish removal solution, and the formulation is then reapplied.

In one embodiment, the amount of the formulation that will be used to treat the infection will be enough to fully cover the infection site and will include a therapeutically-effective amount of the active anti-microbial agent. For example, the anti-microbial agent may be present in an amount between 0.01% and 80% of the weight of the total formulation, and such concentrations will deliver an amount that exceeds minimal inhibitory concentration (MIC) for the targeted organism.

In one embodiment, the formulations of the present disclosure are in any form suitable for application to nail and/or skin tissue, and therefore may further comprise excipients known to prepare, for example, a solution, gel, ointment, paste, paint, bioadhesive, or the like, and/or may be prepared so as to contain liposomes, micelles, and/or microspheres.

The formulations of the present disclosure remain stable and effective after long periods of time, for example one month, 2 months, 3 months, 6 months, one year, two years, or three years without the need for preservatives and/or refrigeration. In one embodiment, the formulations are stable and effective for at least 10 months without the need for preservatives and/or refrigeration. In one embodiment, the formulations are stable and effective for at least 24 months without the need for preservatives and/or refrigeration. The formulations therefore possess a long shelf-life and remain therapeutically active without the need for preservatives and/or refrigeration. In one embodiment, when the organic solvent with tissue permeation ability is dimethyl sulfoxide, the DMSO has a melting point of 19° C., yet the formulations of the present disclosure containing DMSO are stable in liquid form at room temperature (10° C. to 25° C.) for at least 10 months, optionally one year, two years, or three years without the need for preservatives and/or refrigeration.

The formulations of the disclosure are able to dissolve pharmaceutical agents having a wide range of polarities and pKas. For example, the pKa of fluconazole is 1.76, while the pKa of miconazole 6.5.

The following non-limiting examples are illustrative of the present disclosure:

### EXAMPLES

### Example 1

### Anti-Fungal Composition

1 ml of DMSO was mixed with 1 ml of ethoxy diglycol and 8 mls of flexible collodion to form the base formulation. To this base formulation was incorporated 1 g of flucanzole to prepare the pharmaceutical formulation, resulting in an about 10% solution of fluconazole.

### Example 2

### Treatment of Unguis Infection

13 subjects suffering from unguis infections of the fingernails or toe nails were treated using the pharmaceutical formulation of Example 1. Patients applied the formulation to the infected area once or twice a day depending on the severity of the infection.

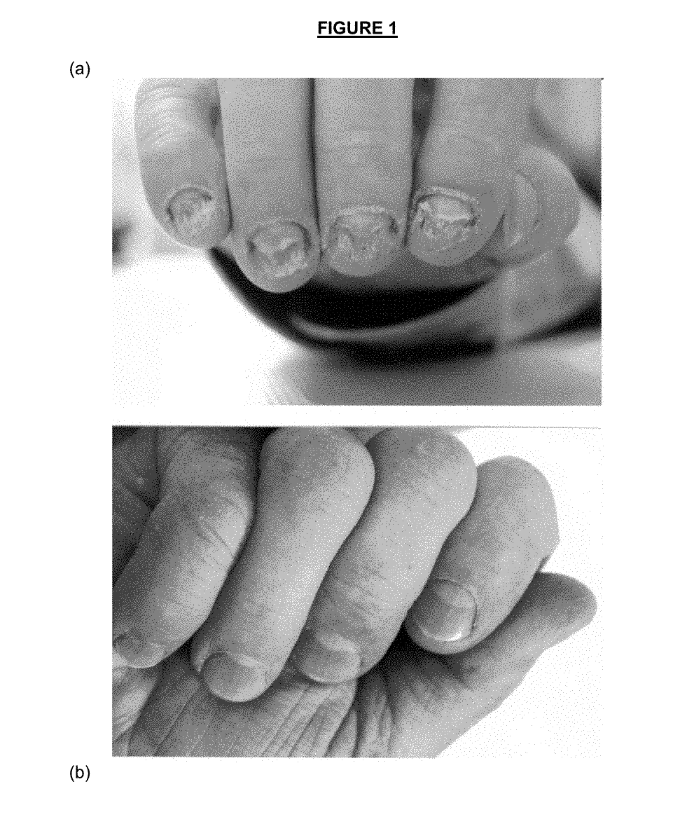

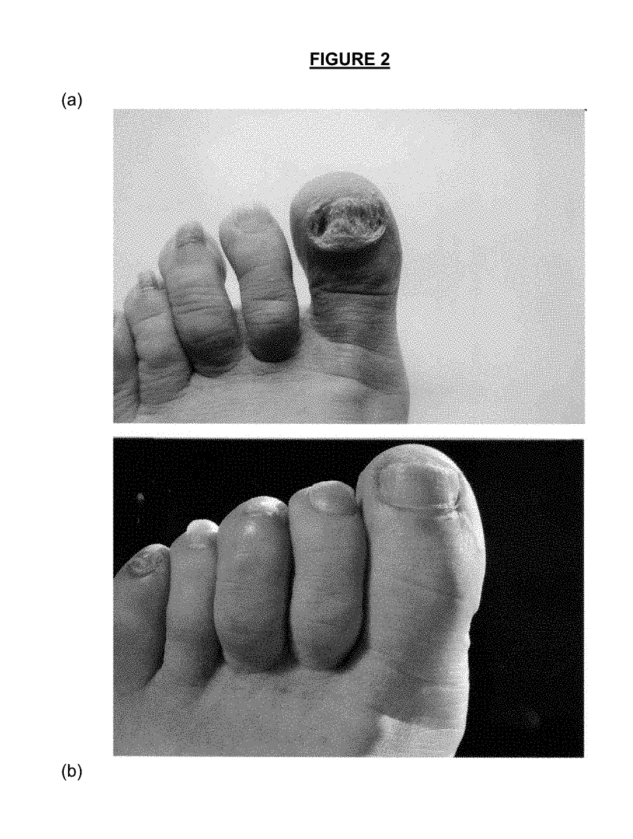

After about 6 months of treatment, patients having fungal infections of the fingernails recovered as seen in FIG. 1. After about 9 months of treatment, patients having fungal infections of the toenails recovered as seen in FIG. 2. None of the subjects reported any systemic or topical adverse effects using the pharmaceutical compositions of the disclosure.

### Example 3

### Treatment of Unguis Infection

13 subjects suffering from unguis infections of the fingernails or toe nails were treated using the pharmaceutical formulation of Example 1. Patients applied the formulation to the infected area once or twice a day depending on the severity of the infection. As shown in Table 1, all patients experienced an improvement in their infection between 2 to 7 weeks after the beginning of treatment. Depending on the severity of the infection, all patients were cured of their infection after being treated for between 12-56 weeks. None of the patients showed a relapse of the infection, or any change in regular laboratory values, or any indication of drug-drug interactions.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Summary of patients treated with Formulation of Example 1

| | | | | | | Weeks |
|---|---|---|---|---|---|---|
| | | | | | Weeks | to |
| | | | | | to | marked |
| | Age/ | Nails | | | 100% | improve- |
| | Sex | Involved | Severity | Dosing | clear | ment |
| 1 | 60/M | Nails 2-5; | All nails | 2 drops of | 42 | 4 |
| | | right hand | infected to | formulation | | |
| | | | root; | twice daily | | |
| | | | subungual | for 1 week; | | |
| | | | infection | after, 2 | | |
| | | | inflamed | drops at | | |
| | | | | bedtime | | |
| 2 | 62/M | Big toe | All nails | Same | 56 | 6 |
| | | and | infected; | | | |
| | | small toe | subungual | | | |
| | | #5 | infection | | | |
| | | | inflamed | | | |
| 3 | 39/M | 2 big toes | ¾ nail | Same | 35 | 4 |
| | | | infected with | | | |
| | | | subungual | | | |
| | | | layer | | | |
| | | | involved | | | |
| 4 | 73/M | 2 big toes | All nail | Same | 49 | 5 |
| | | and small | structures | | | |
| | | toe #5 | infected; | | | |
| | | | yellowish | | | |
| | | | thickening | | | |
| 5 | 68/F | 2 toes #5 | All nail | Same | 24 | 3 |
| | | | structure | | | |
| | | | infected; | | | |
| | | | brittle | | | |
| 6 | 45/F | 1 big toe | All nail | Same | 36 | 4 |
| | | | structure | | | |
| | | | infected | | | |
| 7 | 61/F | 1 big toe | ¾ nail | Same | 41 | 3 |
| | | | structure | | | |
| | | | infected; | | | |
| | | | subungual | | | |
| | | | involved | | | |
| 8 | 72/M | 1 big toe | All nail | Same | 50 | 5 |
| | | and small | structure | | | |
| | | toe #5 | infected; | | | |
| | | | subungual | | | |
| | | | involved | | | |
| 9 | 64/F | 1 big toe | ¾ nail | Same | 15 | 2 |
| | | | structure | | | |
| | | | infected; | | | |
| | | | subungual | | | |
| | | | involved | | | |
| 10 | 12/F | 2 big toes | ¾ nail | Same | 12 | 2 |
| | | and 1 small | infected | | | |
| | | toe #2 | | | | |
| 11 | 44/F | 2 big toes | ¾ of toes | Same | 18 | 3 |
| | | | infected | | | |
| 12 | 38/M | 1 big toe | ¾ nail | Same | 16 | 3 |
| | | | structure | | | |
| | | | infected; | | | |
| | | | subungual | | | |
| | | | involved | | | |
| 13 | 64/M | 1 big toe | all nail | Same | 56 | 7 |
| | | | structure | | | |
| | | | infected/ | | | |
| | | | subungual | | | |
| | | | thickening | | | |

1. A method for the treatment of an Onychomycosis infection of a nailbed, comprising administering to the nailbed a therapeutically effective amount of a non-aqueous topical formulation, the formulation comprising:
(i) an anti-fungal agent present in an amount between 1% and 20% by weight of the total formulation;
(ii) a C₁-C₁₀-alkyl sulfoxide present in an amount between 1% and 20% by weight of the total formulation;
(iii) an organic co-solvent present in an amount between 3% and 30% by weight of the total formulation, wherein the organic co-solvent is a glycol or polyglycol; and
(iv) a film forming agent present in an amount between 40% and 80% by weight of the total formulation, wherein the film forming agent is flexible collodion,
and wherein the non-aqueous topical pharmaceutical formulation is non-desquamating.

2. The method of claim 1, wherein the anti-fungal agent is an allylamine, an imidazole or a triazole.

3. The method of claim 2, wherein the anti-fungal agent is Fluconazole, Tolnaftate, Miconazole, Clotrimazole, Tioconazole, Nystatin, Terconazole, Butoconazole nitrate, Undecylenic acid, Clioquinol, Ciclopirox, Olamine, Econazole nitrate, Triacetin, Flucytosine, Terbinafine or Ketoconazole.

4. The method of claim 3, wherein the anti-fungal agent is Fluconazole.

5. The method of claim 1, wherein the C₁-C₁₀-alkyl sulfoxide is dimethyl sulfoxide.

6. The method of claim 1, wherein the organic co-solvent is ethoxydiglycol, butylene glycol, hexylene glycol or dipropylene glycol.

7. The method of claim 6, wherein the organic co-solvent is ethoxydiglycol.

8. The method of claim 1, wherein the film forming agent is a collodion or flexible collodion.

9. The method of claim 8, wherein the film forming agent is flexible collodion.

10. The method of claim 1, wherein the formulation consists of:
a) an anti-fungal agent present at an amount of 10% by weight of the total formulation;
b) a C₁-C₁₀-alkyl sulfoxide present at an amount of 15% by weight of the total formulation;
c) an organic solvent present at an amount of 10% by weight of the total formulation; and
d) a film forming agent present at an amount of 65% by weight of the total formulation.

11. The method of claim 1, wherein the formulation is for administration once to the infection in every 24-hour period.
